(19) United States
(12) Patent Application Publication
(10) Pub. No.: US 2004/0250815 A1
(43) Pub. Date: Dec. 16, 2004

(54) Heated wire respiratory circuit

(75) Inventors: Kenneth B. Scott, Westerville, OH (US); James B. Gleeson, Columbus, OH (US)

Correspondence Address: KNOBBE MARTENS OLSON & BEAR LLP, 2040 MAIN STREET, FOURTEENTH FLOOR, IRVINE, CA, 92614, US

(73) Assignee: Hudson

(21) Appl. No.: 10/447,178

(22) Filed: May 27, 2003

(51) Int. Cl. F24J 3/00; A62B 7/00; A61M 16/00

(52) U.S. Cl. 128/204.17

(57) ABSTRACT

A respiratory circuit incorporates an insulated heater wire comprising a stranded core of three or more electrically conductive strands of wire.

### BACKGROUND OF THE INVENTION

Ventilator circuits are designed to direct breathing gas to and from a patient, with a ventilator supplying the gas to be breathed under pressure at breathing rates and breath gas volumes prescribed to meet the patient's requirements. Typically, the breathing gas is humidified by a humidifier located at or near the ventilator or respirator whereby the humidified gas must travel substantially the entire length of the inspiratory limb of the circuit. The humidified gas becomes cooled along the inspiratory tubing length resulting in condensation or “rainout” within the tubing. Some respiratory circuits are provided with water traps or other means for removing condensate from the respirator tubing which would otherwise interfere with gas delivery. Alternatively, ventilator or respiratory circuits may be provided with heater wire extending along the interior of the tubing or embedded in or otherwise secured along the wall of the tubing. Examples of such heated ventilator or respiratory circuits are described in U.S. Pat. Nos. 4,682,010, 5,640,951 and 5,537,996. A single heater wire loop having the two ends attached to a plug or connector for supplying electrical current for heating the wire, is commonly used. The wire is provided with a suitable insulation, and is typically assembled by simply inserting the loop of insulated wire into the limb of the circuit. The wire is sometimes secured along the tubing using ties, clips, etc. Although such a heated wire circuit using a loop of generally straight wire is relatively easily assembled, such a design lacks selection, control, or adjustment of the heating capacity of the circuit limb. Because adult, pediatric and neonatal circuits are of different lengths and tubing diameter, each requires a different heating capacity for adequately heating the humidified gas to compensate for heat loss along the tubing length and prevent rainout.

A heated wire respiratory conduit is disclosed in U.S. Pat. No. 6,078,730 using a spirally wound wire. The heater wire is helically wound on a former and then heated to a predetermined temperature sufficient to soften the insulating coating of the wire such that, upon cooling, the heater wire retains its new, helical shape. However, such a process requires placing the wire and former within an oven for a length of time sufficient to soften the insulation which is stiff at ambient temperature, or applying current through the wire, greater than rated, for sufficient time to soften the stiff insulating coating, and thereafter cooling the helically wound wire so that the insulation will set to the desired pitch and diameter. Such a process requires additional equipment and manufacturing time required for heating and processing the wire according to the disclosed method, thereby increasing costs and production time to manufacture and assemble the circuit.

### SUMMARY OF THE INVENTION

The heated ventilator circuit disclosed herein utilizes a heater wire comprising a core of three or more individual strands of electrically conductive wire. The heater wire may be installed in the circuit as a generally straight length or loop of wire or it may be coiled. The heater wire may be single length of wire, generally straight or coiled, or two lengths, for example looped at one end, with the lengths generally straight or coiled. The multiple stranded core has a stiffness capable of maintaining the coiled wire shape formed at ambient temperature, without requiring heating or cooling to form the wire in the desired shape and pitch. The shape of the coiled heater wire, including the pitch and/or diameter, may be selected and formed to achieve a power density necessary for heating different circuits using the same multiple strand insulated wire core. The multiple strand core of three or more wires is also more easily and readily secured to an electrical connector as compared to a single wire strand.

### BRIEF DESCRIPTION OF THE DRAWINGS

FIG. 1 is a sectional view of a limb of a heated wire respiratory breathing circuit incorporating a multiple strand heater wire of the invention;

FIG. 2 is a sectional view of a portion of the insulated heater wire illustrating a preferred embodiment of a insulated core having seven strands of electrically conductive wire;

FIG. 3 is a sectional view of a portion of a ventilator circuit tubing showing a preferred embodiment using helically coiled insulated resistance heater wire;

FIG. 4 illustrates another embodiment of generally parallel helical wires; and

FIG. 5 illustrates a means for securing heater wires near a ventilator end of the breathing circuit.

### DETAILED DESCRIPTION OF PREFERRED EMBODIMENTS

The heater wire disclosed herein comprises an insulated wire with a stranded core of three or more electrically conductive strands of wire. The multiple strand heater wire may be installed in a breathing circuit as a generally straight length of wire, or it may be coiled before it is installed. The number of individual strands in the core is governed by the geometry of the fill patterns that develop stable lays when twisted together. The number-of-strands could be 4, 7, 12, 19, and 37 but 7 or 19 strands are most common. The number of strands is a parameter in determining specific resistance and also influences the flexibility of the wire. The wire core, constructed of multiple strands of a desired or suitable alloy, must have sufficient stiffness to maintain its form when the insulated wire is shaped or wound at ambient temperatures. The construction of a specific wire to obtain a required resistance per foot is influenced by: alloy resistivity, gage and number of strands, temperature coefficient of resistance (TCR), peak heating temperature, power density, and the alloy's tensile strength and modulus. It is preferred to use a wire alloy having a relatively low TCR (e.g., Cu—Ni or 303 stainless steel, etc.) although a high TCR alloy (e.g., Cu) can be used to self-regulate temperature.

FIG. 2 illustrates a sectional view of a portion of an insulated and stranded core, insulation 20 extending around the core 22 of individual wire strands 24. In the drawing, seven strands of wire 24 are used to form the core 22. The use of such multiple strands is beneficial in achieving the desired strength and stiffness of the stranded core, as well as redundancy in the continuity of the electrical heater wire. Multiple strands of electrically resistant wire minimize the risk of arcing or shorting in the event of a break or failure of individual wires. Another advantage of using multiple wire strands is in securing the ends of a length of wire to electrical connectors or contacts. Previously used single strands of wire are more difficult to being terminated adequately to provide continuous electrical connection to such connectors or adapters which are capable of withstanding substantial movement or stress both during assembly as well as use in a hospital or clinical environment. Again, the use of multiple strands provides redundancy in forming a good contact between the wire and the electrical connector as well as presenting a larger core cross-sectional area for making such electrical contact.

Insulation of the wire is preferably a fabric or more preferably a thermoplastic, and is not stiff at ambient temperature, but rather soft and flexible enough so that it does not substantially resist or overcome the stiffness of the stranded core and its ability to adequately maintain a desired helical shape formed at ambient temperature. Suitable plastic insulating materials include the amorphous thermoplastics having low modulus and durometer properties. Examples of such materials include polyvinyl chloride, polystyrene, polyethylene, polypropylene, etc. Filler materials as well as additives, plasticisers, modifiers, etc. may be used to control the stiffness. Any number of polymers or thermoplastics considered to be crystalline may be used if so modified, which will be understood by those skilled in the art. Again, the aforesaid thermoplastics or polymers are given only by way of example and are not intended to be limiting to insulative materials which may be used in the wires described herein. It is important that the insulation material used on the core of wires will permit the stiffness of the stranded core of wires to achieve a helical shape, pitch and/or diameter, formed at ambient temperature, and will maintain the shape throughout the ambient temperature manufacture and assembly, and thereafter at the temperatures and conditions at which the respiratory circuit is used and exposed.

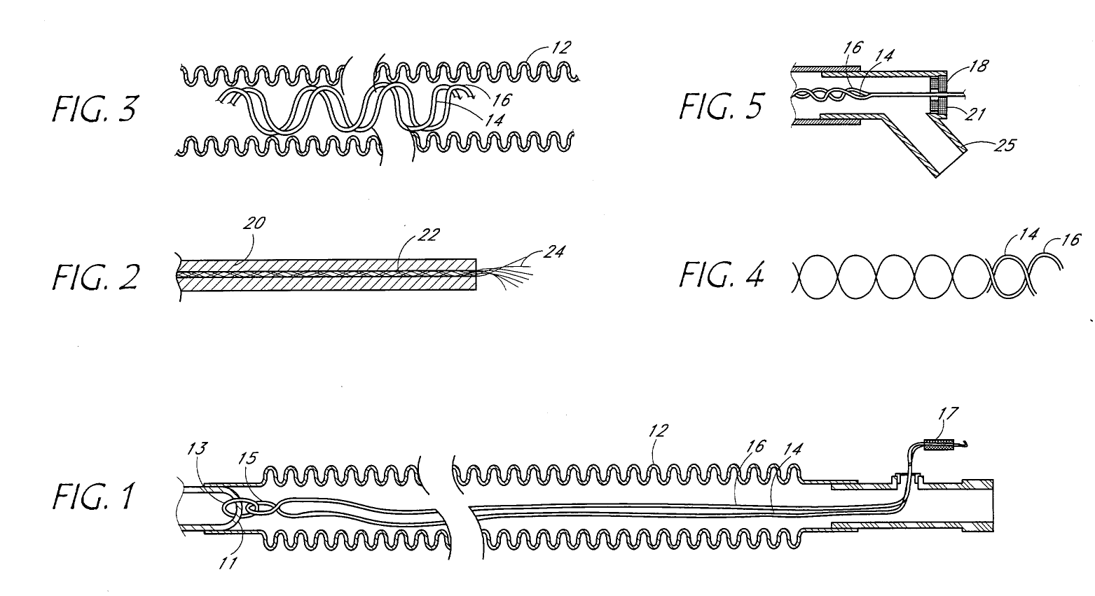

In a first embodiment, the insulated, multiple strand heater wire is used without being coiled. As shown in FIG. 1, the wire is installed as a loop of generally parallel insulated stranded wires 14 and 16 along the interior of tubing 12. The wire is simply positioned along the tubing interior and looped around a connector 13 which is secured to bar 11 at or near the distal end of the leg or length of tubing. Any equivalent means for securing the end of the wire loop may be used. For example, the wire loop may be directly attached to a peg, hook, hanger, or other suitable member or means formed in the tubing or to a connector 13, as shown, for securing the looped end of wire. The heater wire may be installed with as much or as little slack desired. The connector may be an elastomeric band, or a plastic tie loop, or the like. Other means for securing the wire along the tubing interior may also be used, and that shown is by way of example. An electric connector 17 is attached to the two ends at the ventilator end of the looped wire. Alternatively, a single length of wire may be used, with the circuit completed to the two opposite ends exterior to the tubing.

In a second embodiment the heater wire is coiled prior being installed along the interior of the tubing. Observing FIGS. 3 and 4, the heater wire circuit used in the respiratory device is preferably a pair of generally parallel helically shaped insulated stranded wires 14 and 16. As used herein, the terms “parallel” or “generally parallel” are intended to indicate that adjacent portions of the insulated wire follow a similar helical shape. The preferred embodiment is a “parallel” path, typically fashioned by both wires having the same pitch and diameter (similar to the geometry of a double start lead screw) that minimizes the likelihood of crossed wires and the resulting localized hot spots. Alternatively, the coiled wire may be a single length of helically shaped wire instead of the two wires as shown. The length of wire for the heater circuit can be wound on a mandrel (at ambient conditions) to form the “generally parallel” insulated wire having substantially the same pitch and diameter. An example of the winding process could use a single loop of the heater wire, fixing the mid-point of the loop to the mandrel, and rotate the mandrel to lay both legs of the circuit at a fixed pitch along the length of the mandrel. An alternative method of winding would fix one end of the wire to the mandrel, rotate the mandrel to lay the first leg of the circuit at a fixed pitch along the length of the mandrel, securing the free end of the wire to the mandrel, and reversing the direction of rotation, to lay the second leg of the circuit at a fixed pitch to return along the length of the mandrel. Both winding processes are equivalent in final product and differ in complexity and efficiencies of manufacture. A heater wire circuit can be constructed with other wire geometries (e.g., unequal pitch of wires 14 and 16) and, accordingly, the watt density must be selected to accommodate the closeness or proximity of the wires.

The coil or helix is formed by shaping or otherwise forming or winding a length of generally straight wire on a mandrel or former. The helical shape of the wire may be obtained by winding or shaping the generally straight length of wire on a mandrel, preferably generally circular around its outer surface, such as a rod or tubular shaped mandrel. The wire is shaped at ambient temperature, requiring no heating or cooling during the shaping or forming operation. Ambient temperature may be any atmospheric temperature of the surrounding area, such as room temperature, for example, between about 50° F. to about 100° F., or so. It is not intended to exclude or be limited to any specific ambient temperature, surrounding atmospheric temperature, room temperature, whether it be conditioned by refrigeration, or not, in which the manufacturing and assembly of insulated wires and such respiratory products may be carried out. However, elevated temperatures, such as required by heating a thermoplastic insulation to a temperature above ambient at which the thermoplastic material becomes softened or otherwise substantially changes its elasticity or stiffness different from those properties at ambient temperature, are not used in the processing and treatment of the heater wire as described herein.

The helically coiled heater wire described herein may be easily adjusted for different specific selected power densities to meet different circuit requirements. For example, adult circuits having large diameters and long tubes have relatively high heat losses, and thus have greater heating requirements as compared to shorter and smaller diameter pediatric and neonatal circuits. A length of heater wire having a smaller pitch, i.e., a greater number of helical turns or coils per unit of length, has a greater power density or watt density at a given current as compared to wire having a greater pitch or fewer turns per unit of length, assuming the same coil diameter. As previously described, the heater wire of the present invention is capable of substantially maintaining a helical shape and specific pitch in a static or unstressed condition when formed at ambient temperature. Such a pitch may be quite suitable for achieving the desired watt/ft. power density, for example, for use in an adult ventilator circuit having relatively high heat requirements. Moreover, the heater wire described herein is capable of being readily reshaped at ambient temperature to a second pitch at a static, unstressed condition. For example, where a heater wire as described above is initially helically formed at a first pitch of 0.25 inch (4 turns per inch), it may be readily reshaped at ambient temperature, for example, by simply stretching or pulling the wire at ambient temperature to form a helical shape having a pitch of 0.50 inch (2 turns per inch) which it maintains in a static, unstressed condition, suitable for a reduced power density heating requirement. Observing FIGS. 1 and 4, the pitch of the embodiment illustrated in FIG. 1 is about twice the number of turns per unit of length as compared to the pitch of the embodiment illustrated in FIG. 4. Again, this change in pitch may be accomplished readily at ambient temperature by direct forming at this shape or by reshaping, such as stretching or compressing, the coil to modify the pitch as desired. Coiling the wire also controls or limits tension of the wire thereby maintaining integrity of the insulation, and enhancing safety of the circuit. Thus, the effective stiffness of the wire of any given or selected wire geometry, determined by number of strands, type of wire and insulation, can be controlled by limiting pitch of the coiled wire. Thus, coiling of the wire results in a soft wire spring to provide the desired effective tension limiting feature.

The diameter of the helical shape of the wire may be initially formed so as to accommodate any diameter tubing. The mandrel or former on which the wire is wound will determine the diameter of the helical coil. Generally, the diameter of coiled wire will be somewhat greater than the diameter of the mandrel due to expansion of the wire following release of the winding tension or force used. The degree of expansion will depend on a number of factors such as wire stiffness, composition and flexibility of the insulation, winding tension, diameter of the mandrel, etc. The coil diameter will be selected to readily fit into the tubing. Larger diameter coils may also influence the pitch of the coil, to minimize the length of wire used for a selected power density. Although large pitch reduces wire length requirements, desirable for cost reduction, at a specific diameter a larger pitch reduces the tension limiting capacity, or spring rate, of the coil. Thus, the diameter of the coil may also be selected to achieve a desired spring rate. The distal end of the coiled wire may be secured as shown in FIG. 1 previously discussed.

FIG. 5 illustrates an alternative means for securing the heater wire at a machine end or ventilator end of a respiratory circuit. As shown, a snap-on, in-line grommet 18 is used in port 21 of adapter 25 and through which heater wires 14 and 16 are pulled. The size of the grommet opening, although restricted, is sufficient to allow slippage of the wires where necessary for limiting or reducing tension. The grommet may also be rotated to provide desired orientation of the wires during and following installation.

By way of example, a heater wire as described herein was prepared using a wire coil having seven strands of Cu—Ni alloy wire, a core diameter of 0.019 inch and insulated with PVC having an OD of 0.070 inch. The insulated wire was helically wound on a ⅛-inch diameter mandrel at 4 turns per inch at ambient room temperature of 60′-70° F. The fabricated helical coil had a pitch of 0.25 inch and a mean diameter of 0.25 inch. Other heater wire pitch may be achieved by stretching or pulling the wire.

What is claimed is:

1. A respiratory breathing circuit comprising:
one or more limbs of hollow tubing having a heater wire extending interiorly therein along a substantial portion of the length of one or more limbs of said hollow tubing,
said heater wire comprising an insulated core of three or more strands of electrically conductive wire.

2. A respiratory breathing circuit of claim 1 wherein said insulated core has 7 strands of electrically conductive wire.

3. A respiratory breathing circuit of claim 1 wherein said insulated core has 19 strands of electrically conductive wire.

4. A respiratory breathing circuit of claim 1 wherein said heater wire comprises a helically wound insulated core having stiffness capable of substantially maintaining said insulated wire in helical shapes formed at ambient temperature.

5. A respiration breathing circuit of claim 1, 2, 3, or 4 wherein said heater wire comprises a single length of said electrically conductive wire.

6. A respiratory breathing circuit of claim 1, 2, 3 or 4 wherein said heater wire comprises a loop of two generally parallel lengths thereof.

7. A respiratory breathing circuit comprising:
one or more limbs of hollow tubing having a heater wire therein comprising:
an insulated core of three or more electrically conductive strands of wire,
wherein the heater wire is shaped in the form of a helix at ambient temperature,
and wherein said core of wires has a stiffness sufficient to maintain a helical shape formed by shaping the insulated core of wires at ambient temperatures.

8. A respiration breathing circuit of claim 7 wherein said heater wire comprises a single length of said electrically conductive wire.

9. A respiratory breathing circuit of claim 7 wherein said heater wire comprises two substantially parallel lengths of said helically shaped wire.

10. A respiratory breathing circuit of claim 7 or 9 wherein said helically shaped wire has a first pitch and/or diameter in a static, unstressed condition at ambient temperature and is capable of being shaped at ambient temperature to a second pitch and/or diameter in a static, unstressed condition.
